(19) United States
(12) Patent Application Publication
(10) Pub. No.: US 2025/0295850 A1
(43) Pub. Date: Sep. 25, 2025

(54) TRANSDERMAL DRUG ADMINISTRATION DEVICE

(71) Applicant: Société industrielle de Sonceboz S.A., Sonceboz-Sombeval (CH)

(72) Inventors: Jocelyn Staub, Charquemont (FR); Thierry Martin, Mont de Vougney (FR); Nicolas BaÏlo, Nidau (CH)

(21) Appl. No.: 18/860,781

(22) PCT Filed: Apr. 26, 2023
(86) PCT No.: PCT/EP2023/060977

§ 371 Date: Oct. 28, 2024

(30) Foreign Application Priority Data
Apr. 26, 2022 (FR) FR2203900

(51) Int. Cl. A61M 5/14; A61M 5/142; A61M 5/145; A61M 5/24

(52) U.S. Cl. CPC A61M 5/1413; A61M 5/14244; A61M 2005/14208; A61M 2005/14268; A61M 2005/14573; A61M 2005/2488; A61M 2205/8206

(57) ABSTRACT

A transdermal drug administration device comprises: a housing; a battery; an injection system; a movable connection part for providing fluid connection between the injection system and a reservoir containing an active ingredient intended to be administered; a pump system, the pump system being motorized so as to cause a fluid to circulate within a fluid network of the device; and an electronic system for controlling the driving of the pump system, the electronic system being provided with a mechanical switch for powering the pump system with a battery, wherein the movable connection part is moveable relative to the housing and the reservoir, the movable connection part jointly controlling the fluid supply of the pump and the power supply of the electronic system during the movement, at least part of the movement being performed by a human mechanical action.

### CROSS-REFERENCE TO RELATED APPLICATIONS

This application is a national phase entry under 35 U.S.C. § 371 of International Patent Application PCT/EP2023/060977, filed Apr. 26, 2023, designating the United States of America and published as International Patent Publication WO 2023/209018 A1 on Nov. 2, 2023, which claims the benefit under Article 8 of the Patent Cooperation Treaty of French Patent Application Serial No. FR2203900, filed Apr. 26, 2022

### TECHNICAL FIELD

The present disclosure relates to the field of external transdermal drug delivery systems, incorporating a pump system and a transdermal administration system. Typically, the system is integrated into a housing whose interior maintains the sterility of the fluid, self-contained part, which adheres to the patient's abdomen or chest or other area and delivers the substance to the patient via a cannula that is inserted into the patient subcutaneously.

### BACKGROUND

The medical treatment of several diseases requires continuous infusion of drugs, via subcutaneous and intravenous injections, particularly for drugs containing large molecules that cannot be digested when administered orally, such as insulin, biological medicines or biosimilars

Patients suffering from immuno-oncology type chronic diseases or else requiring recurrent post-operative treatment, need bolus injections of drugs, which are usually administered by nursing staff. The quantity of injections of these drugs may vary from a few milliliters to several tens of milliliters. As the flow rate is capped in order to be tolerated by the patient, injection time increases proportionally with the volume injected and has a direct impact on the occupancy level of the nursing staff. Automatic drug injection pumps have been developed on the one hand to free up nursing staff time when administration via the pump needs to be supervised by trained personnel, and on the other hand, to allow patients to self-administer drugs safely thanks to a simplified automatic system, giving them daily autonomy and drastically reducing the frequency with which they need to visit the hospital.

Basal and bolus volumes must be administered at precise doses according to individual prescriptions. As a result, drug injection pumps need to be highly reliable, ensuring that patients and caregivers can accurately monitor the correct dose.

These pumps are also used for the injection of biological medicines, or active anticancer ingredients, requiring shorter injection times, from a few tens of minutes to a few hours, at less frequent weekly or monthly intervals.

To avoid problems of re-sterilization of ambulatory devices intended for home treatment, single-use devices are preferred. The simplicity of the components of the device is, therefore, essential to ensure a sale price that is not prohibitive.

Usually, the patient fills the reservoir with the active ingredient, for example, from a vial, attaches the needle and administration tube to the reservoir outlet orifice, then inserts the reservoir into the pump housing. After purging the air from the reservoir, tube and needle, the patient inserts the needle assembly, penetration element and cannula at a selected location on the body and removes the penetration element. To avoid irritation and infection, the subcutaneous cannula must be replaced and discarded with the empty reservoir.

Alternatively, the patient or caregiver may use an additional transfer station to automatically transfer the contents of the vial into the internal reservoir of the medical device. To do this, the user generally needs to successively connect the vial (and vial adapter if it is not already integrated into the transfer station) on the one hand, and the medical device on the other hand, to the transfer station, then start the transfer sequence.

These devices are equipped with an internal energy source that powers the internal members when the drug is injected into the patient. As these devices can be stored for long periods, it is crucial to preserve the power source during this time and to power the system only when required.

U.S. Patent Application Publication No. US2013253427 describes a drug dispensing device, which comprises a key validation part and a security part. The key validation part is configured to interact with a key part of another component of the drug dispensing device during assembly of the drug dispensing device. To support the use of corresponding components, the security part is configured to have a different impact if the key part corresponds to the key validation part, compared to the case where the key part does not correspond to the key validation part.

U.S. Patent Application Publication No. US2020023123 describes a drug administration system and components thereof. The system may comprise a body-mounted pump device and a secondary unit. The body-mounted pump device may comprise a reservoir and a fluid pathway. The reservoir may be designed to hold a liquid medicine. The secondary unit may be releasably coupled to the body-mounted pump device. The secondary unit may be designed to receive a pre-filled cartridge containing a liquid medicine, expel the liquid medicine from the pre-filled cartridge, and dispense the liquid medicine to the reservoir of the body-mounted pump device via the fluid pathway. This is a solution consisting of two assembled parts, namely a body-mounted pump device 301 and a secondary unit 305, to form the injection arrangement, contrary to the disclosure. In addition, there is never any provision for a receiving part for the mobile vial. The receiving part is formed by a fixed cavity. This solution is not compatible with fluid communication via a vial. It requires a special vial with a perforable base. This solution also requires the bottom to be pierced with two needles; one for air intake to compensate for the volume of liquid transferred.

U.S. Patent Application Publication No. US2020146938 describes a disposable fluid transfer and mixing device that comprises an injector support surface for receiving an injection device thereon. A lyophilized drug vial, a diluent vial and a syringe are also attached to the device. The device comprises fluid passages and a manual valve that can be manipulated so that the syringe can be used to transfer diluent from the diluent vial to the lyophilized drug vial, for reconstitution of the drug. The valve may also be designed so that the reconstituted drug is transferred from the lyophilized drug vial to the injection device. An automated transfer and mixing device receives an injection device, a lyophilized drug vial and a diluent vial and transfers the diluent from the diluent vial to the lyophilized drug vial and shakes or vibrates the lyophilized drug vial to reconstitute the drug.

U.S. Patent Application Publication No. US2020368447 describes a housing and an activation button assembly movably mounted thereon and able to be moved from an unactuated to an actuated position. A cartridge door is configured to receive therein a cartridge containing a substance to be dispensed, and is movably mounted on the injector housing between open and closed positions. A deflectable interference member is mounted inside the injector housing and, in a rest position, blocks the movement of the activation button assembly from the non-actuated to the actuated position thereof. The activation button assembly is configured to activate the injector when in the actuated position, and can be moved from the non-actuated position to the actuated position only upon deflection of the interference member from its rest position. The cartridge door, when in its closed position with the cartridge arranged inside, deflects the interference member out of its rest position.

Devices known in the prior art propose to power the device upon removal of the cover protecting the part of the device to be applied to the patient and whose sterility must be ensured until the last moment. This solution is satisfactory in its context but is incompatible with a device having a removable vial, which potentially requires the internal reservoir to be filled prior to injection. Indeed, filling the internal reservoir is a slow operation, which is performed by a pumping mechanism that requires an electrical power supply. Based on this known technique, the patient would have to remove the cover in order to switch on the pumping system, immediately apply the device to their skin and wait for the pumping operation to finish before the injection takes place. This means unnecessary immobilization of the patient during an automatic operation for which his or her presence is not essential.

Removable vial devices known in the prior art do not offer solutions for preventing unintentional powering on of the device by linking it to a crucial step that is not subject to handling errors. While prior art devices can satisfactorily secure the internal reservoir filling step after the vial has been inserted, this is ensured by the use of sensors that must be supplied with power. Thus, in this context, the prior art does not propose to guarantee the connection of the device to the energy source thereof only during an unavoidable step of the injection procedure that cannot be unintentionally triggered.

In particular, U.S. Patent Application Publication No. US2013253427 describes a solution with a system for checking the conformity of the vial/device pair using an electromechanical coding system. This solution implies that the electronic circuit must first be switched on before the signals supplied by these indexes can be used. As with other solutions in the prior art, there is a risk of untimely electrical power consumption, with a consequent reduction in service life and autonomy.

### BRIEF SUMMARY

To remedy the shortcomings of the prior art, the present disclosure relates, in its most general sense, to a transdermal drug administration device

In some embodiments, the transdermal drug administration device of the present disclosure comprises:
a. a housing,
b. a battery,
c. an injection system,
d. a movable connection part for providing fluid connection between the injection system and a reservoir containing an active ingredient intended to be administered,
e. a pump system connected to the movable connection part and to the injection system by virtue of flexible pipes so as to produce a fluid network, the pump system being motorized so as to cause a fluid to circulate within this network,
f. an electronic system for controlling the driving of the pump system, the electronic system being provided with a mechanical switch for powering the pump system with battery,
the movable connection part has at least one degree of freedom relative to the housing on one side and the reservoir on the other in order to allow a movement, the movable connection part jointly controlling the fluid supply of the pump and the power supply of the electronic system during the movement,
at least part of the movement of the movable connection part being performed by a human mechanical action,
the movable connection part comprising a means for fluid connection between the reservoir and the pump on the one hand providing disconnection at rest and fluid communication during the movement,
the movable connection part comprising a means for activating the electrical power supply of the electrical and electronic components of the device, providing disconnection at rest and supplying power during the movement.

In one variant, the movable connection part is also designed to receive the reservoir in the form of a vial, which is removable from the transdermal drug administration device, the movable connection part being moved in the at least one degree of freedom, when receiving the reservoir.

In addition, the reservoir is connected to the movable connection part by means of an adapter part, connected beforehand to the reservoir, the movable connection part and the adapter part having complementary central pipes that fit together to form a sealed connection.

In addition, the complementary central pipes can be used to create a sealed connection by means of a tapered fitting.

In particular, the tapered fitting created by the complementary central pipes may be of the “luer” type.

In one variant, the movement path of the movable connection part, according to the at least one degree of freedom, has a notch at the end of travel to lock the movable connection part in the final position, the notch requiring extra torque to be passed and reach the final position, with the switch being activated in the final position.

In particular, the extra torque required to pass the notch and reach the final movement position of the movable connection part, is greater than the nominal tightening torque for screwing the adapter part to the movable connection part, thus guaranteeing the seal of the central pipe connection.

In another variant, the movable connection part has a cylindrical body with a tapping, with the adapter part having a hollow cylindrical protuberance into which the central pipe extends, the hollow cylindrical protuberance having a thread on its inner surface that engages with the tapping to ensure the sealed fitting of the central pipes by screwing the adapter part to the movable connection part, the sealing being guaranteed beyond a nominal tightening torque.

In particular, the degree of freedom of the movable connection part for activating the switch may be a rotational degree of freedom achieved by guiding the movable connection part in the lower casing of the housing, the movement path of the movable connection part being angular, and in that the movable connection part has a partial annular protuberance axially extending the cylindrical body over a limited angular portion, the partial annular protuberance being capable, at the end of travel, of activating the switch located in radial proximity to the movable connection part.

In one variant, the movement path of the movable connection part is traversed by the rotational movement leading to the screwing of the adapter part to the movable connection part.

In one variant, the movable connection part has two cylindrical axial protuberances extending from the cylindrical body, these axial protuberances extending into the lower casing of the housing and simultaneously coming into abutment against beads during the course of the movement path of the movable connection part, the beads being excrescences of flexible beams integral with the lower casing, the abutment position of the axial protuberances against the beads being able to be exceeded by elastic deformation of the flexible beams, being elastically deformable, to reach the final movement position of the movable connection part, the notch thus being formed by the elastic deformation of the flexible beams.

In all variants, the adapter part can be screwed to the movable connection part by the user.

In particular, the deformation and slackening of the flexible beams, when the user applies excess torque to pass the notch, provides haptic information signaling activation of the switch and, therefore, of the device.

In another variant, the movable connection part has a radial protuberance and a partial annular protuberance, the final position of the movable connection part being achieved by the radial protuberance and the partial annular protuberance being respectively brought into contact against complementary stops on the lower casing of the housing.

In another variant of an injection device with a pre-filled internal reservoir, the movable connection part is equipped with a hollow needle connected to a flexible pipe, the hollow needle being able to be inserted into the reservoir during a translational movement of the movable connection part to reach a final state.

In addition, the translational movement is induced by a spring.

More particularly, in the initial state, the movable connection part has no translational degree of freedom, but only one translational degree of freedom, and in that the spring is in a compressed state.

More precisely, a first rotational movement of the movable connection part is performed by a mechanical action of the patient to bring the movable connection part into an intermediate state in which it acquires the translational degree of freedom allowing the hollow needle to be inserted into the reservoir.

### BRIEF DESCRIPTION OF THE DRAWINGS

For a better understanding of the present disclosure, and to show how

embodiments thereof may be implemented, reference will now be made, by way of example, to the appended drawings wherein:

FIG. 1 is a top view of the locking device of the insertion mechanism in the initial state;

FIG. 2 is a top view of the locking device of the insertion mechanism in the final state;

FIG. 3 is the needle insertion mechanism alone in a perspective and partial cross-sectional view;

FIG. 4 is a perspective view of the locking mechanism associated with the pump system in a partial cross-section and housed in the lower casing of the housing;

FIG. 5 is a side view of the pump system associated with the locking mechanism;

FIG. 6 is a perspective view of the complete fluid system integrated in the lower casing of the housing;

FIG. 7 is a side cross-sectional view of the pump;

FIG. 8 is a top view of the pump body from which the valve plunger emerges;

FIG. 9 is a cross-sectional front view of the pump;

FIG. 10 shows two pump hole shapes for connecting pipes and the fluid flow cross-sections obtained for different valve plunger positions;

FIGS. 11 through 13 are perspective views of the fluid system compatible with an external reservoir for different steps of reservoir insertion, with FIG. 11 showing all the components before assembly, FIG. 12 showing the reservoir positioned in the adapter part, and FIG. 13 showing the reservoir and adapter part assembly assembled with the movable connection part and in the final position;

FIG. 14 is a cross-sectional side view of the adapter part and of the movable connection part;

FIG. 15 is a side view of the movable connection part;

FIGS. 16 and 17 are top views of the movable connection part integrated into the lower casing of the housing in the initial and final position of the activation travel, respectively;

FIGS. 18 and 19 are cross-sectional top views of the movable connection part integrated into the lower casing of the housing in the initial and final positions of the activation travel, respectively;

FIG. 20 is a cross-sectional top view of a variant of the movable connection part integrated into the lower casing of the housing in the initial position;

FIG. 21 is a perspective view of a second variant of the pre-filled internal reservoir device;

FIG. 22 is a top view of the second variant with a pre-filled internal reservoir in the initial state;

FIG. 23 is a cross-sectional side view of the second variant with a pre-filled internal reservoir in the initial state;

FIG. 24 is a top view of the second variant with a pre-filled internal reservoir in the final state; and

FIG. 25 is a cross-sectional side view of the second variant with a pre-filled internal reservoir in the intermediate state.

### DETAILED DESCRIPTION

Multifunction operation and plunger rod-controlled release of the insertion mechanism

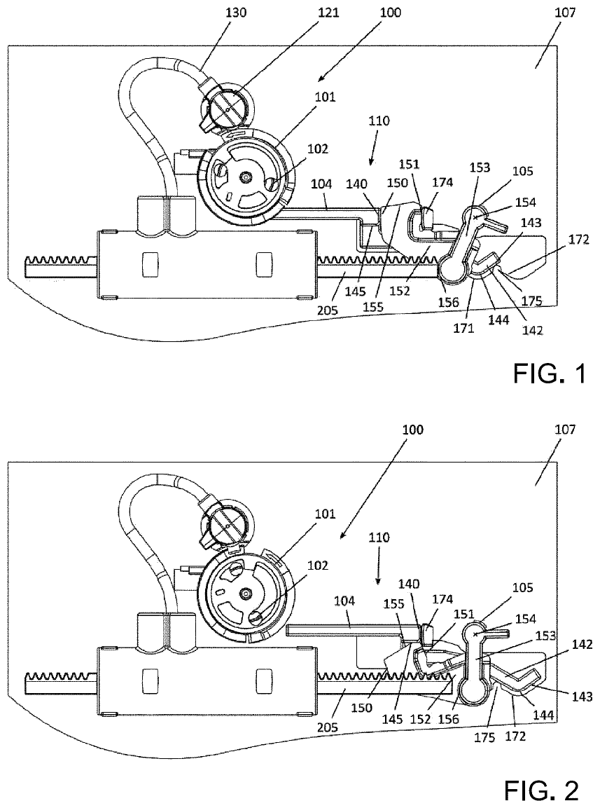

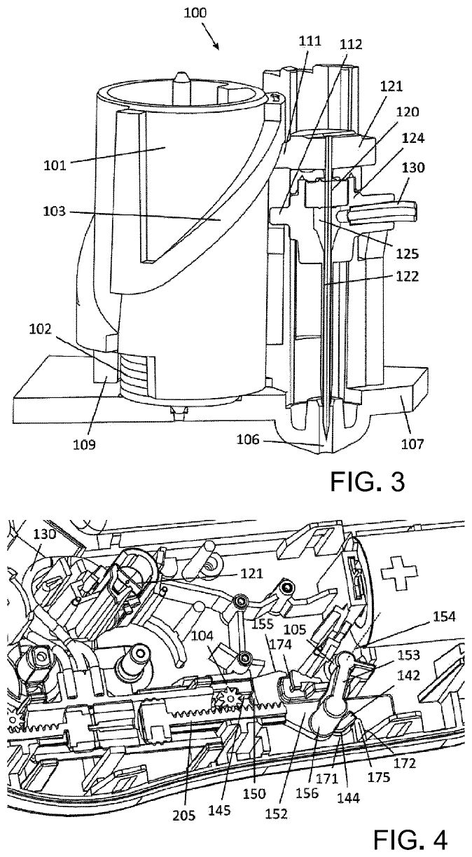

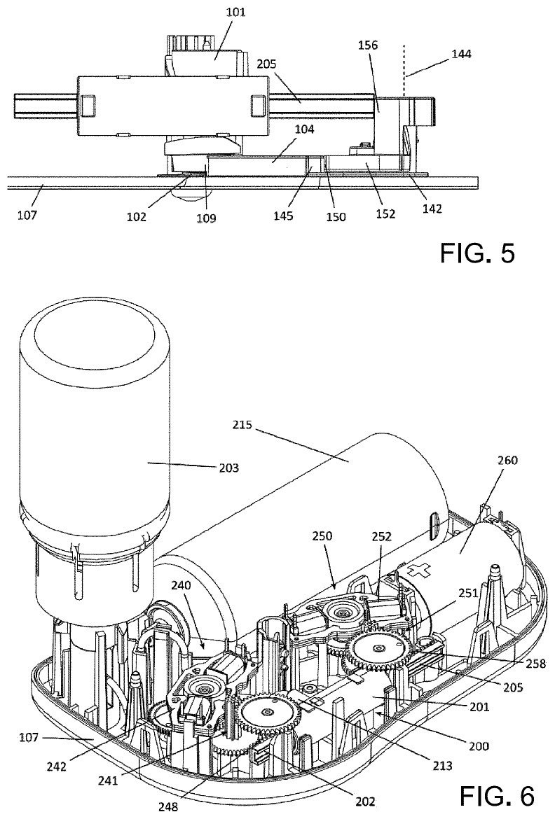

FIGS. 1, 2, 3, 4, and 5 show a first embodiment of the locking device of the needle insertion mechanism according to the disclosure, with FIG. 1 showing the needle insertion mechanism only, as described in international patent application WO2018141697A1. FIGS. 2 and 3 show the needle locking and insertion mechanism in a top view, with FIG. 2 showing the device in the initial state, known as “armed,” and FIG. 3 showing the mechanism after release and needle insertion. FIG. 4 shows the needle locking and insertion mechanism in a perspective view and FIG. 5 shows the needle locking and insertion mechanism, without the cam element, in a side view.

The injection system (100) includes a needle insertion mechanism consisting of a cam element (101) and shown in more detail in FIG. 1, which is a partial cross-sectional view. This cam element (101) has a rib (103) whose advantageous profile engages with the protuberances (111, 112) of a needle holder (121) and a cannula holder (124) allowing the insertion of the needle (120) and the flexible cannula (122), then the withdrawal of the needle (120) only, in a rotational movement of less than 360° of the cam element (101). The cannula holder (124) has a conduit for receiving the tube (130) carrying the fluid to be injected through the cannula (122), this conduit being connected to the cannula (122) by an internal cavity (125). Insertion of the needle (120) and the cannula (122) is guided through a through-hole (108) in the lower casing (107) of the housing. The rotational movement of the cam element (101) is induced by a spiral spring (102), preloaded in the initial state, the cam element (101) being held in position by a locking device (110) described more particularly in FIGS. 2, 3, 4, and 5. The insertion sequence of the needle (120) is triggered by releasing the locking device (110), allowing free rotation of the cam element (101) and, therefore, insertion of the needle (120) by virtue of the unloading of the spiral spring (102). It should be noted that this sequence of movements is irreversible, and that when completed, the spiral spring (102) is in the unloaded state and opposes movement of the cam element (101) in the opposite direction to that used for insertion of the needle (120) and cannula (122). Thus, once the sequence has been completed, the needle (120) is retracted inside the medical device and cannot be deployed again, leaving the device in a state that avoids any risk of injury to the user. The use of a retractable needle (120) housed within a flexible cannula (122) does not limit the present disclosure and is especially useful for guaranteeing the sterility of the fluid conduit as long as the needle is located within the cannula. The skilled person could easily imagine other alternatives for creating the fluid interface between the device and the patient.

More particularly, the locking device comprises a translational element (104) and a rotational element (105), both of which are guided by the lower casing (107) of the housing in respective translational and rotational movements.

The translational element (104) has an arm (140), the distal end of which rests on a lateral surface (109) of the cam element (101), more visible in FIG. 5, and the proximal end of which rests on a surface (150) of the rotational element (105), and an elastic protrusion (142) ensuring that the locking device (110) is held in its final position after release, by engaging with the groove (172) in the lower casing (107) of the housing. The rotational element (105) has a surface (151) opposite the surface (150), this opposite surface (151) resting on a protuberance of the lower casing (107) of the housing. Thus, in the locked position, the engagement of the translational element (104) and of the rotational element (105) allows the entire preload force of the spiral spring (102), exerted on the cam element (101), to be transmitted directly to the lower casing (107) of the housing, thus preventing any possible movement. This also ensures a longer service life of the system during a storage period, while the use of direct load transfer from the housing avoids the risk of creep of the parts.

The locking device (110) can be released by moving the rotational element (105). The rotational element (105) has a body (152) with a lever arm (153) for guiding the body (152) in rotation about the axis (154). Rotation of the rotational element (105) causes the surface (150) and the opposite surface (151) to slide over the opposing surfaces (141, 174) belonging respectively to the translational element (104) and to the lower casing (107) of the housing.

When the rotation of the rotational element (105) is sufficient for the surface (150) to no longer be in contact with the surface (141) of the translational element (104), the locking mechanism (110) is released and the translational element (104) performs a translational movement induced by the preload force of the spiral spring (102). During the translational movement of the translational element (104), the arcuate profile (143) of the elastic protrusion (142) slides along the grooves (171, 172) of the housing, causing the end of the elastic protrusion to move orthogonally to the translational movement of the translational element. When the apex (144) of the arcuate profile (143) passes the separation (175) between the two grooves (171, 172), the translational element (104) is no longer in contact with the cam element (101), but the translational movement continues along the groove (172) thanks to the release of the elastic energy accumulated by the elastic protrusion (142) as it moves along the first groove (171), finally arriving at the stable equilibrium position shown in FIG. 3. During the translational movement of the translational element (104), the lower surface (145) of the translational element (104) slides over an upper surface (155) of the rotational element (105) so as to force the rotational element (105) to move into abutment against a protuberance (173) on the lower casing (107) of the housing. The complementary profile of the surfaces (145, 155) advantageously allows the translational element (104) to continue the translational movement following this abutment, while at the same time blocking the rotational movement opposite the abutment in the final position as shown in FIG. 3.

Thus, once the locking device has been released, the elastic protrusion ensures a safe final position in which the translational element (104) and the rotational element (105) are immobilized, even in the event of sudden movement or impact on the medical device.

Before the locking device is released, the translational element (104) and the rotational element (105) are held in position by virtue of the friction of the surface (150) and the opposite surface (151) on the surfaces (141, 174) respectively. Rotation of the rotational element (105) and hence release of the locking device is achieved by pressing the end of the pump plunger (205) against a circular surface (156) of the lever arm (153). In the embodiment shown, this pressure is achieved on an end part of the movement path of the pump plunger (205), advantageously allowing the pumping function to be performed before the locking device is released, allowing, for example, the fluid to be injected to be transferred from an external reservoir to an internal reservoir before the device is placed on the patient. During this operation, the stroke of the pump plunger may be easily limited by software.

Of course, once the locking device (110) has been triggered by a first movement of the pump plunger (205) on the extreme part of the stroke thereof, the pump plunger (205) can benefit from the full stroke for the pumping function and thus increase, for each pump cycle, the volume transferred from the internal reservoir to the patient.

Note that the pump plunger (205) only ensures the rotational movement of the rotational element (105) until the locking device (110) is released, as described above. In order to ensure free movement of the plunger on this end stroke, it is necessary, after the locking device (110) is released, to maintain the rotational element (105) in a safety position as permitted herein by its engagement with the translational element (104).

### Pump Body with Oblong Holes

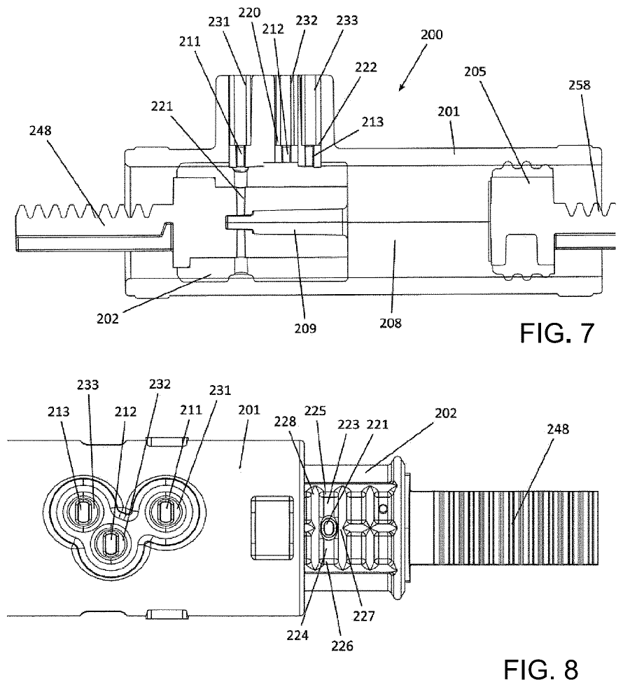

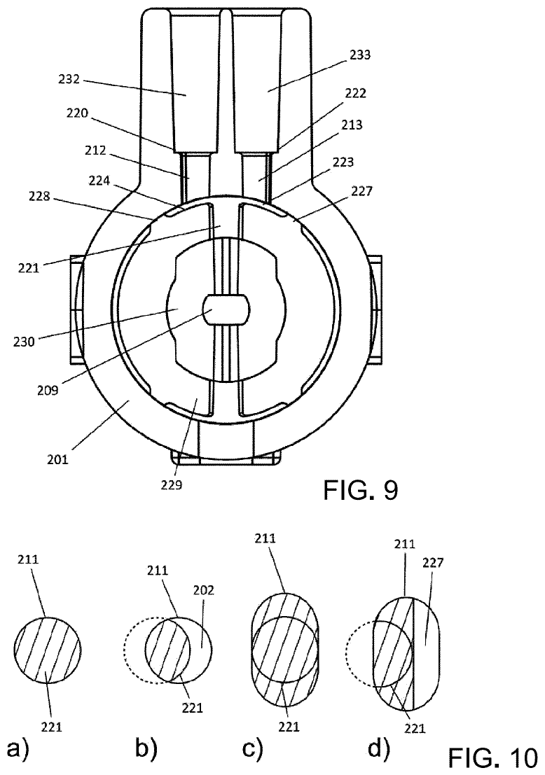

One embodiment of the pump system (200) is shown in FIGS. 6 to 10. FIG. 6 shows the pump system (200) integrated in the lower casing (107), connected to the reservoir (203) of the fluid to be extracted and to the internal reservoir (215). FIG. 7 shows a cross-sectional side view of the pump system (200) revealing the fluid circuit. FIG. 8 shows a side view of the pump body (201) from which the valve plunger (202) emerges. FIG. 9 shows a radial cross-section of the pump system (200), with the pump body (201) cut in two axially offset planes to reveal the ports (212 and 213) simultaneously. FIG. 10, Panels a) to d) highlight the geometry of the three ports (211, 212, 213) of the pump body (201) and their advantage over those used in international patent application WO2018141697A1.

More particularly, and as shown in FIGS. 6 and 7, the pump is composed of a pump body (201) and two plungers, a pump plunger (205) and a valve plunger (202), defining therebetween an internal cavity (208) of variable volume. These two plungers are respectively connected to a rack (248, 258), which can be set in axial motion by a drive system (240, 250) composed of a mechanical motion reducer (241, 251), the input of which is coupled to an electric motor (242, 252). Fluid transfer within the internal cavity (208) is made possible by the engagement of multiple ports (211, 212, 213) with an internal channel (209) of the valve plunger (202) opening at one end into the internal cavity (208) and terminated by a pump orifice (221) at the other end thereof. Precise axial positioning of the valve plunger (202) allows selective engagement of one of the ports (211, 212, 213) with the pump orifice (221).

In the example shown in FIG. 6, port (211) is connected to the reservoir (203) of the fluid to be extracted, for example, a vial, port (212) is an outlet port and is connected to the injection system, port (213) is connected to the internal reservoir (215) of the medical device. Port (211) and port (213) can be used alternatively as input ports or output ports. A pump outlet port is understood to be a port from which the fluid flows exclusively so as to leave the internal cavity (208) of the pump. In the initial state, the valve plunger (202) and pump plunger (205) are in frontal contact or are only slightly apart, so that the internal cavity (208) has a low air volume.

Two injection sequences are preferred without limiting the disclosure. Either using the internal reservoir or by direct injection.

The first injection sequence involves transferring the liquid from the vial to the internal reservoir, then transferring the liquid from the internal reservoir to the injection system. The first pumping sequence is then broken down into the following steps:
synchronously moving the valve plunger (202) and the pump plunger (205), by their respective drive systems (240, 250), to align the pump orifice (221) with the port (211) connected to the internal reservoir (215),
moving the pump plunger (205) only, by virtue of the drive system (250), so as to increase the volume of the internal cavity (208) located between the valve plunger (202) and the pump plunger (205), thus creating a negative pressure leading to liquid being drawn from the reservoir (203) into the internal cavity (208) through the internal channel (209),
once the internal cavity is completely filled, moving the plungers (202, 205) synchronously, using their respective drive systems (240, 250), so as to align the pump orifice (221) with the port (213) connected to the internal reservoir (215),

moving in translation the pump plunger (205), only, by the drive system (250) thereof, so as to reduce the volume of the internal cavity (208) in order to expel all the fluid contained in the internal cavity (208) through the internal channel (209) and towards the internal reservoir (215),

once the liquid has been expelled, repeating the preceding steps successively until all the liquid contained in the reservoir (203), or a predetermined quantity, is transferred to the internal reservoir (215).

Once the fluid has been completely transferred to the internal reservoir (215), injection into the patient can begin. The first step is, therefore, to trigger the needle insertion system as described, for example, in international patent application WO2018141697A1. Once the cannula has been inserted into the subcutaneous tissue, a new pumping sequence takes place according to the following steps:
synchronously moving the valve plunger (202) and the pump plunger (205), via their respective drive systems (240, 250), to match the pump orifice (221) with the port (213) connected to the internal reservoir (215),
moving the pump plunger (205) only, by means of the drive system (250), so as to increase the volume of the internal cavity (208) located between the valve plunger (202) and the pump plunger (205), thus creating a negative pressure leading to liquid being drawn from the internal reservoir (215) into the internal cavity (208) through the internal channel (209),
once the internal cavity is completely filled, moving the plungers (202, 205) synchronously, using their respective drive systems (240, 250), so as to align the pump orifice (221) with the port (212) connected to the injection system,
moving in translation the pump plunger (205), only, by the drive system (250) thereof, so as to reduce the volume of the internal cavity (208) in order to expel all the fluid contained in the internal cavity (208) through the internal channel (209) and towards the injection system.
once liquid expulsion is complete, repeating the previous steps successively until all the liquid contained in the internal reservoir (215), or a predetermined quantity, has been transferred to the patient.

The second injection sequence eliminates the need to transfer fluid to the internal reservoir. The first step is, therefore, to trigger the needle insertion system as described, for example, in international patent application WO2018141697A1. Once the cannula has been inserted into the subcutaneous tissue, a pumping sequence takes place according to the following steps:
synchronously moving the valve plunger (202) and the pump plunger (205), by their respective drive systems (240, 250), to align the pump orifice (221) with the port (211) connected to the reservoir (203),
moving the pump plunger (205) only, by virtue of the drive system (250), so as to increase the volume of the internal cavity (208) located between the valve plunger (202) and the pump plunger (205), thus creating a negative pressure leading to liquid being drawn from the reservoir (203) into the internal cavity (208) through the internal channel (209),
once the internal cavity is completely filled, moving the plungers (202, 205) synchronously, using their respective drive systems (240, 250), so as to align the pump orifice (221) with the port (212) connected to the injection system,
moving in translation the pump plunger (205), only, by the drive system (250) thereof, so as to reduce the volume of the internal cavity (208) in order to expel all the fluid contained in the internal cavity (208) through the internal channel (209) and towards the injection system.
once liquid expulsion is complete, repeating the previous steps successively until all the liquid contained in the reservoir (203), or a predetermined quantity, is transferred to the patient.

These two injection sequences do not limit the disclosure, and other alternatives are contemplated. In particular, the pump system (200) may be used for drug reconstitution functions, for example, to reconstitute a lyophilized drug and a diluent. The internal reservoir (215) could then be a cartridge pre-filled with a diluent, and a pump sequence could be performed to transfer this diluent to a reservoir (203) containing a lyophilized drug in order to dissolve it. Once the lyophilized drug has dissolved, reverse transfer from the reservoir (203) to the internal reservoir (215) is carried out for subsequent injection into the patient. It is also conceivable to carry out several successive transfers, partial or total, between the reservoir (203) and the internal reservoir, as this may facilitate dissolution of the lyophilized drug in the diluent.

Alternatively, the external reservoir (203) could be dispensed with altogether, the internal reservoir (215) being a cartridge pre-filled with the fluid to be injected. In this case, the fluid is transferred from the internal reservoir (215) to the internal cavity and then to the injection system. This sequence of steps is repeated as many times as necessary.

As can be seen more particularly in FIG. 8, ports (211, 212, 213) are staggered, with ports (211 and 213) lying in the same longitudinal plane, port (212) being radially offset and lying axially between the two ports (211 and 213). This staggered configuration is particularly advantageous for limiting the travel required by the valve plunger (202) to select one of the ports (211, 212, 213), as this procedure must be repeated several times during an injection cycle. The axial proximity of the ports (211, 212, 213) has a direct influence on the durability of the battery (260) and, therefore, on the overall performance of the device. The downside is that the axial dimension of the ports is limited and, therefore, the axial positioning of the valve plunger (202) must be more precise, as incorrect positioning results in a reduction in the fluid flow cross-section. Depending on the viscosity of the fluid, this can lead to significant pressure losses and, therefore, a reduction in fluid flow.

It is assumed from this principle that the positioning of the orifice of the valve plunger of the pump opposite one of the ports is a critical point in the current state of the art, as embodied in international patent application WO2018141697A1. Ensuring a minimum fluid flow cross-section in the event of axial “mispositioning” of the plunger has the advantage of making the system more stable and robust and limiting pressure losses, thus guaranteeing the accuracy of the flow rate, quantity of drug injected into the patient, and the injection time. The disclosure overcomes this problem by describing two improvements that can be used independently or in combination.

FIGS. 8 and 9 highlight one of these improvements. The staggered arrangement of the ports (211, 212, 213) results in a tangential misalignment of these holes with respect to the pump orifice (221) of the valve plunger (202). Thus, so as not to reduce the pump flow rate, the orifice (221) of the valve plunger (202) has a tangential flare (223, 224) on either side of the radial end thereof facing the ports (211, 212, 213). These flares are delimited by tangential beads (225, 226) and axial beads (227, 228) made of a flexible and deformable material, such as silicone or a soft thermoplastic, to match the cylindrical inner surface of the pump body (201) and ensure sealing. To this end, the valve plunger has a hard core (230), wherein the internal channel (209) connected to the pump orifice (221) is formed, this hard core (230) being overmolded by an elastomer (229) to form the tangential flares (223, 224). FIG. 9 shows a radial cross-section of the valve body, with the left and right halves of the valve not in the same axial plane, but in two half-planes respectively in the middle of the port (212) and of the port (213). This cross-section shows the correct alignment between the tangential flares (223, 224) and the ports (212, 213) offset tangentially relative to the pump orifice (221) of the valve plunger (202).

The second improvement in valve plunger position tolerance is shown in FIGS. 7, 8 and 10. In particular, the ports (211, 212, 213) of the pump body (201) are extended by tapered recesses (231, 232, 233) for coupling with the cylindrical flexible pipes (261, 262, 263 (not shown)), connecting elements of the fluid system while providing a radial stop for these pipes by virtue of a shoulder (220, 222). Consequently, the diameter of the ports (211, 212, 213) must be smaller than the outer diameter of the flexible pipes and ideally larger than the inner diameter of the flexible pipes so as not to limit the flow rate. Although the shoulder secures the mechanical assembly, it is not, however, necessary for it to be present around the entire circular periphery of the flexible pipes. Thus, the ports (211, 212, 213) may advantageously have an oblong periphery to increase the cross-section opposite the valve plunger orifice (202) while at the same time providing a mechanical stop for the flexible pipe.

Oblong periphery is understood to mean that the cross-section of the ports (211, 212, 213), as shown in FIG. 8, is not disc-shaped but has a different length in the axial and tangential directions. In the example shown in FIG. 8, and in Panels c) and d) of FIG. 10, the cross-section is a groove in the tangential direction, terminated by two half-cylinders. However, the disclosure is not limited to this type of shape, but includes any type of elongated section, such as a rectangle or ellipse. Also, the long length is not limited to the tangential direction but can also be realized in the radial direction and will be selected according to the arrangement of the ports (211, 212, 213).

FIG. 10 shows different positioning scenarios for the valve plunger (202), with Panels a) and b) representing the case of a pump orifice (221) and a port (211) both with a disc section, as realized in patent application WO2018141697A1. Panel a) shows a cylindrical port (211) with a cross-section of 0.196 mm² for a diameter of 0.5 mm and being perfectly aligned with an identical pump orifice (221), the fluid flow cross-section is represented by cross-hatching. Panel b) shows the evolution of the hatched cross-sectional area when there is an axial misalignment between the orifice (221) and the port (211), it can be seen that an offset of 0.1 mm results in a remaining cross-sectional area of 0.122 mm², that is, a reduction of over 35%. The oblong shape of the port (211) according to the disclosure is shown in Panels c) and d), and offers a fluid cross-sectional area of 0.321 mm² in the same axial space when combined with an identical orifice or a disc orifice with a tangential flare as shown. Panel d) shows the evolution of the hatched cross-sectional area in the event of incorrect axial positioning of the pump. Note that for an offset of 0.1 mm, the cross-section decreases to leave a passage of 0.268 mm². Note also that up to an axial offset of 0.2 mm, that is, a cross-sectional area of 0.198 mm², the flow cross-section remains greater than or equal to that obtained for a disc section port of the same axial dimensions. This means that even with an offset of 0.2 mm corresponding to a limit tolerance, the shape of the present disclosure ensures a fluid flow cross-section equivalent to the cylindrical shape of a 00.5 mm tube as achieved in the prior art. The shape of the present disclosure therefore enables precise positioning at ±0.2 mm without impacting the pressure losses in the system.

It should be noted that the disclosure is not limited to an oblong shape but may be extended to any shape that increases the fluid flow cross-section while ensuring the radial stop of the pipes connected to the pump body.

### Activation by Reservoir Insertion

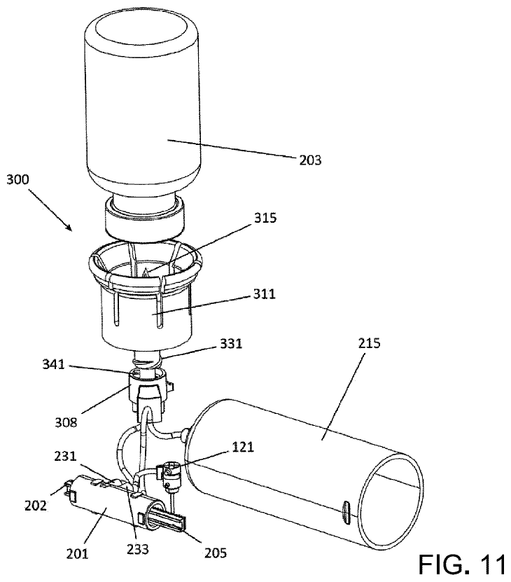

The medical device according to one variant of the disclosure, to be considered in FIGS. 11 to 20, can interface with an external reservoir (203), such as a vial. To simplify the use of the device and the reliability thereof, the vial must be coupled with an adapter part (311) designed to fit into a movable connection part (308) of the medical device, having complementary shapes. In the example shown in FIG. 5, the adapter part (311) has a hollow needle (315) designed to pierce the septum of the reservoir (203), or vial, and withdraw the fluid contained therein, the hollow needle (315) being connected to a central channel of the adapter part (311). This operation is carried out before the medical device is activated and the configuration of the septum and needle are such that only fluid flow through the internal channel is possible, thus preventing air from entering the reservoir (203) and, therefore, preventing the spontaneous flow of the fluid contained in the reservoir (203). Activation of the medical device is achieved by inserting the adapter part (311) into the movable connection part (308). To this end, the adapter part (311) and the movable connection part (308) can be screwed into each other so that complementary central pipes (320, 321) on both parts engage to provide a sealed flow channel for conveying fluid between the vial and the medical device. In the example shown, the central pipes (320, 321) engage by means of a tapered fitting to form a luer-type connection, but the disclosure extends to any type of connection compatible with medical applications. When the adapter part (311) is screwed into the movable connection part (308), the torque exerted by the adapter part (311) on the movable connection part (308) drives the latter in rotation in order to activate the electronic system (310) and authorize the transfer of the drug to the internal reservoir as described in international patent application WO2018141697A1. Once the drug has been transferred, the user can disconnect the adapter part (311) by unscrewing it, this movement then returning the movable connection part (308) to the initial position thereof and simultaneously deactivating the electronic system (310). During the activation and deactivation sequence of the system by the means described, the user may be informed about the status of the medical device by haptic or visual signals, such as vibrations or the powering of LEDs. The triggering of the signals can be directly activated by the user's actions on the device.

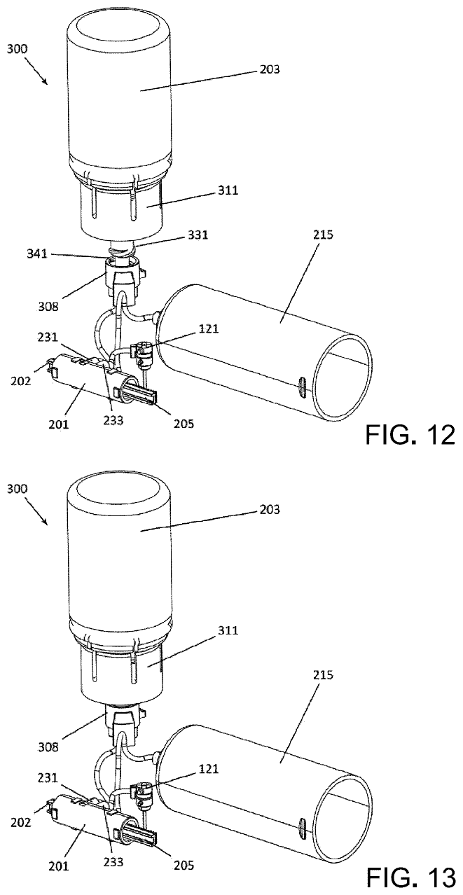

FIGS. 11 to 13 show the isolated fluid system (300) of one embodiment of the present disclosure. FIGS. 11 and 13 are perspective views showing the various steps involved in connecting an external reservoir (203), with FIG. 11 showing an exploded view of the various unconnected elements, FIG. 12 showing the connection of the reservoir (203) to the adapter part (311), and FIG. 13 showing the connection of the reservoir (203) and adapter part (311) assembly to the movable connection part (308). These various figures also show the other components of the fluid system, that is, the fluid part of the pump system (200), the internal reservoir (215) and the needle holder (121) of the injection system, these various elements being connected by flexible pipes, one end of which is inserted into tapered recesses (231, 233) in the pump body (201). Fluid is directed to either element by virtue of the movements of the valve plunger (202) and pump plunger (205) described in this application. In particular, FIG. 11 shows the presence of a hollow needle (315), designed to pierce the septum of the external reservoir to make a sealed connection between the external reservoir (203) and the pipework of the fluid system (300). The assembly shown in FIG. 13 is obtained, from FIG. 12, by screwing together the adapter part (311) and the movable connection part (308) by engaging a thread (331) and a tapping (330).

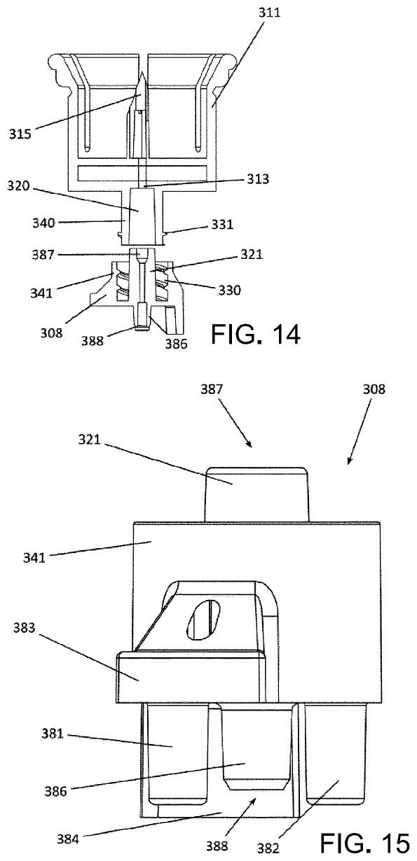

FIG. 14 shows a cross-sectional view of the adapter part (311) and the movable connection part (308) before assembly, in order to better visualize the fluid connection between these parts and their connection. In particular, complementary central pipes (320, 321) belonging respectively to the adapter part (311) and to the movable connection part (308) provide fluid connection between the fluid outlet port (313) of the adapter part (311) and the inlet port (387) of the movable connection part (308). The hollow needle (315) of the adapter part (311) has an internal channel terminating in the fluid outlet port (313), which opens into the central pipe (320). The movable connection part (308) is formed by a cylindrical body (341) having an axially open cavity into which the central pipe (321) opens out, the inner cylindrical surface of the cavity having the tapping (330) engaging with the thread (331) of the adapter part (311). At the other axial end thereof, the movable connection part (308) has a hollow axial protuberance (386) with a slightly tapered periphery so that it can easily be inserted into a flexible pipe, to ensure the transfer of fluid to the pump body. The movable connection part is thus axially traversed by a conduit opening on the one hand, onto the inlet port (387) at one axial end of the central pipe (321), and on the other hand, onto an outlet port (388) located at the end of the axial protuberance (386).

The central pipes (320, 321) are advantageously tapered in shape, known as a “luer taper,” allowing a sealed coupling in compliance with ISO 80369, when sufficient contact force is ensured. The interlocking of the complementary central pipes (320, 321) and the application of the force required for sealing are provided by virtue of the engagement of a thread (331) complementary to a tapping (330), respectively made in the hollow cylindrical protuberance (340) of the adapter part (311) and in the cylindrical body (341) of the movable connection part (308), surrounding the central pipes (320, 321). Thus, once the adapter part (311) and the movable connection part (308) are assembled, any fluid entering through the hollow needle (315) is fed to the outlet port (388) without any possible leakage. The movable connection part (308) ensures and secures the supply of fluid from the pump system (200) to the reservoir (203) containing the drug.

It should be noted that the arrangement of this connection does not limit the disclosure, as the skilled person can easily imagine alternatives for pipes of two complementary parts in a sealed manner by a screwing method.

FIG. 15 gives a more detailed view of the movable connection part (308) shown in FIGS. 11 to 14. In the upper part of this figure, the central pipe (321) emerging from the hollow cylindrical body (341) is visible, featuring the tapping for screwing to the adapter part (311). Also shown is the cylindrical body (341) for guiding the rotation of the movable connection part (308) by engaging with an annular protrusion of the lower casing (107) of the housing. Finally, in order to ensure the rotational stop once the adapter part (311) has been fully screwed into the movable connection part (308) by the user, two cylindrical axial protuberances (381, 382) open out from the cylindrical body (341), these axial protuberances (381, 382) are located radially on either side of the axial protuberance (386) and are designed to extend into the lower casing (107) of the housing, so as to engage with an elastically deformable part, to ensure that the final angular position of the movable connection part is maintained. The final angular position is obtained by abutment of a radial protuberance (383), radially opening out from the lower axial end of the cylindrical body (341), against a complementary abutment of the lower casing of the housing. Finally, the presence of a partial annular protuberance (384) may be noted, axially extending the cylindrical body (341) over a limited angular portion. This partial annular protuberance (384) is called an activation arm and allows the medical device activation switch to be engaged or released depending on the angular position of the movable connection part (308). The partial annular protuberance (384) also has a concentric cylindrical inner surface with a cylindrical protrusion of the lower casing of the housing, to ensure that the movable connection part (308) is rotatably guided again.

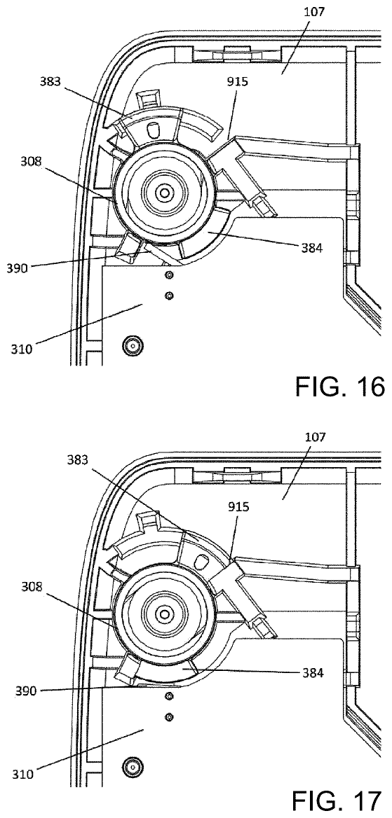

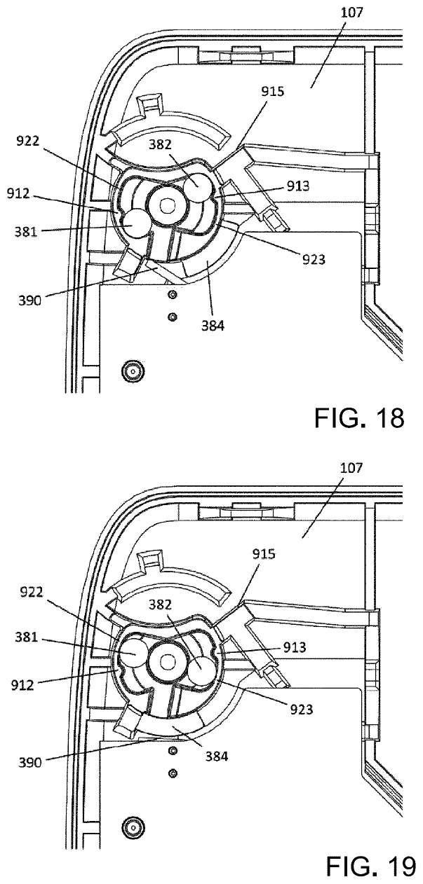

FIGS. 16 and 18 show the movable connection part (308) installed in the lower casing (9) of the housing in the initial state thereof, that is before insertion of the reservoir fitted with its adapter part. FIG. 16 showing a view from an axial angle and FIG. 18 showing a cross-sectional view, at the level of the axial protuberances (381, 382), from the same angle. During tightening, the adapter part (311) is inserted axially into the movable connection part (308), with the movable connection part remaining stationary, until the central pipes are in coaxial contact so as to be wedged. Therefore, if additional torque is applied to the adapter part (311), this torque is transmitted to the movable connection part (308) whose rotation is stopped by the axial protrusions (381, 382) bearing against the beads (912, 913) of the lower casing (107) of the housing. The beads (912, 913) are protrusions located in flexible beams (922, 923) that can be elastically deformed by the axial protrusions (381, 382), when a threshold torque is applied, allowing the movable connection part to rotate until the radial protuberance (383), together with the partial annular protuberance (384), abuts against complementary stops (915, 916) of the lower casing (107) of the housing. This final position is shown in FIGS. 17 and 19, corresponding to FIGS. 16 and 18, respectively. It may be noted that the axial protrusions (381, 382) can be deformed alternatively or in addition to the flexible beams (922, 923) to allow rotation of the movable connection part (308) when a torque greater than the threshold torque is applied to the latter.

As the movable connection part (308) rotates, the partial annular protuberance (384) presses against the lever of the activation switch (390) of the medical device arranged on the electronic system (310). At the end of movement, the radial protuberance (383) and the partial annular protuberance (384), when they come into respective contact with the complementary stops (915, 916), absorb any excess torque supplied by the user to the movable connection part (308) preventing this excess torque from impacting on the axial protuberances (381, 382) thus avoiding damage thereto.

It should be noted that the deformation torque required to rotate the movable connection part (308) is greater than the minimum torque required to guarantee the seal of the connection according to ISO standard 80369. This deformation serves as haptic feedback to the user to indicate successful installation of the reservoir (203) and activation of the medical device.

It should also be noted that the final angular position is not irreversible. If the user applies a torque of the same amplitude as that deployed to activate the device, but in the opposite direction, the flexible beams (922, 923) can once again be elastically deformed by the axial protrusions (381, 382), allowing the movable connection part to rotate back to return to the initial state shown in FIGS. 16 and 18. This operation is accompanied by the disarming of the switch (390), indicating to the medical device that the vial is no longer engaged. The user can continue unscrewing the adapter part (311) to remove the reservoir (203).

This activation system thus offers a function that guarantees the user a monitored and secure insertion of the vial to limit any risk of misuse. Indeed, as the fluid connection of the pump system (200) with the reservoir (203) and the powering of the electronic system (310) supplying the pump system (200) are both carried out by the same movable connection part (308), during the same operation, it is impossible for the device to initiate a pumping and injection sequence without the cartridge being correctly inserted, just as it is equally impossible for fluid contact to be made and injection to be triggered a long time later, for example, several days, ruling out any risk of mishandling or contamination.

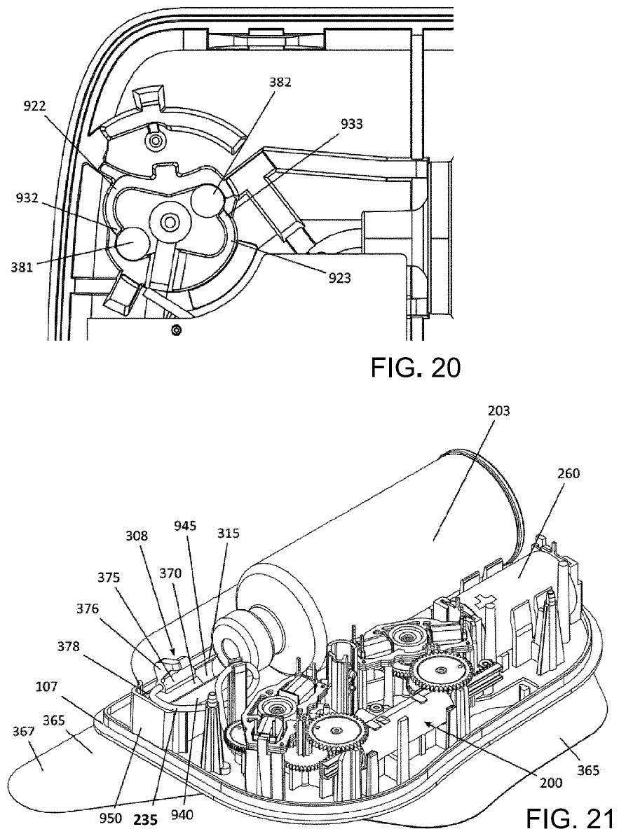

FIG. 20 shows a variant of the flexible beams (922, 923), which no longer have a central bead but instead have an arcuate shape ending in a free end (932, 933). FIG. 20 shows a cross-sectional view of the movable connection part (308) at the level of the axial protuberances (381, 382) and installed in its initial state, that is, before the reservoir (203) fitted with its adapter part (311) is inserted. In this initial position, the axial protrusions (381, 382) respectively abut the free ends (932, 933). Rotation of the movable connection part (308) when the vial is screwed in is enabled by elastic deformation of the flexible beams (922, 923) by centrifugal displacement of their free ends (932, 933). Once sufficient deformation has been applied, the axial protuberances (381, 382) can be accommodated at the base (943) of the flexible beams causing the elastic stress to release and the free ends (932, 933) to return to their initial position. This configuration provides a hard point in the vial insertion procedure in order to provide haptic feedback to the user once elastic release of the flexible beams (922, 923) is complete. This configuration is advantageous compared with the previous method as it allows asymmetry between the effort required to screw in the vial and the effort required to unscrew it. In fact, elastic deformation of the beams is much easier when carried out from the base of the beam towards the end thereof than vice versa. By suitably selecting the curvature of the flexible beams (922, 923) and the profile of the surface of the free ends (932, 933) in contact with the axial protuberances, it is possible to obtain screwing torques that are very different from unscrewing torques, for example, more than twice as high. This is particularly useful for ensuring perfect completion of the unscrewing sequence of the reservoir (203). In fact, the screwing torque required to overcome the hard point formed by the flexible beams is very close to the torque required to tighten the adapter part (311) in the movable connection part (308). An unscrewing torque to pass the hard point of the same magnitude, could cause the adapter part (311) to unscrew from the movable connection part (308) before passing the hard point generated by the flexible beams, leaving the movable connection part (308) in the final state and, therefore, the device activated while the reservoir is removed.

### Activation by Tearing Off the Protective Cover

In one particular embodiment shown in FIGS. 21 to 25, the transdermal drug administration device is fitted with an internal reservoir (203) that is solely pre-filled beforehand with the drug to be injected. In fact, the embodiment previously presented and equipped with an external reservoir involves an operation of filling the internal reservoir, which requires the pump system. This operation may be slow and time-consuming for medical staff, but may also be subject to handling errors. An alternative is, therefore, to dispense with these steps in order to offer the patient a device that already contains the appropriate drug and that minimizes the number of manipulations required. The device is loaded beforehand by a qualified person and kept sterile until it is used by the patient. To limit the risk of contamination, the drug is stored in a sealed internal vial until a hollow needle (315) penetrates the septum of the reservoir.

As in the previous embodiment, this hollow needle (315) is connected via a flexible pipe (235) to the pump system (200), which is, in turn, connected to the injection system (100) (not shown). This embodiment also features a movable connection part (308) for fluidly connecting the pump system (200) to the reservoir (203), and for powering the electronic system (310) supplying the pump system (200).

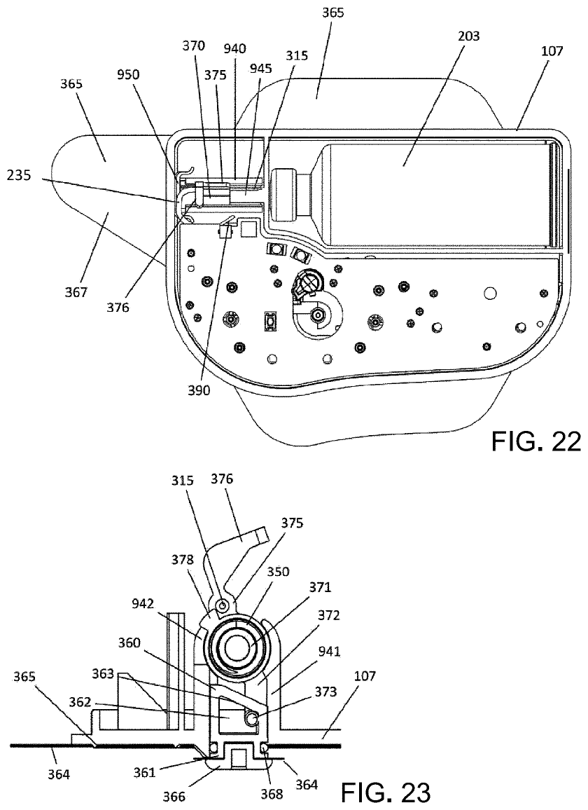

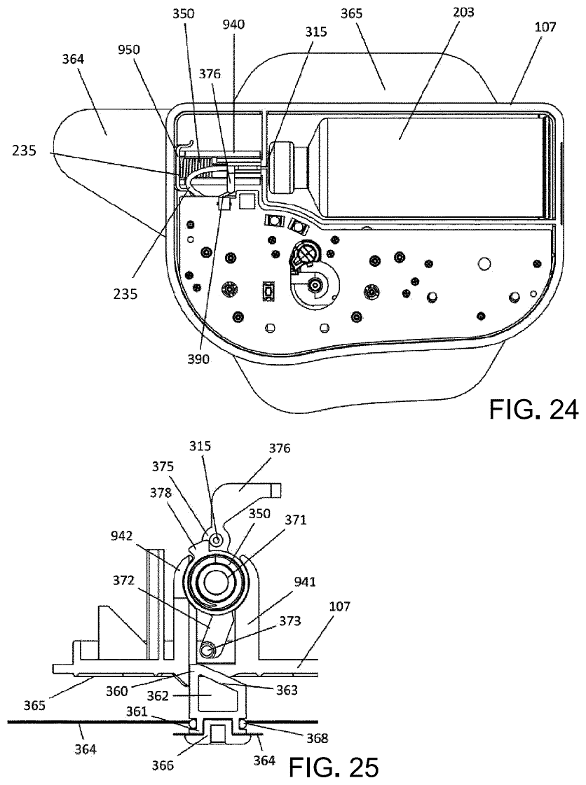

However, this method differs from the embodiment shown in FIGS. 6 to 20 in that the reservoir (203) has no adapter part (311) and that the hollow needle (315) is directly supported by the movable connection part (308). As shown more particularly in FIGS. 22 to 25, this movable connection part (308) has a complex movement broken down into a first rotational movement, performed by a mechanical action of the user, followed by a translational movement performed by the relaxation of a spring (350) preloaded during the device storage period. FIGS. 22 and 24 show the injection device seen from above and with the cover open, respectively in the initial and final positions of the activation procedure. FIGS. 23 and 25 show the injection device seen from the side, perpendicular to the hollow needle (315), and with the cover open, respectively in the initial position and in an intermediate position of the activation procedure.

In this embodiment, the movable connection part (308) is in the form of a cylindrical body (370) guided in a complementary guide support (940) integral with the lower casing (107) of the housing to allow a sliding pivot-type connection. For the purposes of this section, the regions of the cylindrical body (370) and guide support (940) directly opposite the head of the reservoir (203) will be referred to as proximal parts, and the regions on the opposite side, along an axis of cylindrical symmetry of the reservoir (203), will be referred to as distal parts.

The cylindrical body (370) is provided with an annular section to form an internal cylindrical cavity (371) opening out at the distal end of the cylindrical body (370), this internal cylindrical cavity accommodating a spring (350). The cylindrical body (370) is equipped, at the proximal base of the annular section, with a radially extending leg (372), the leg allowing, in the initial position, axial abutment against a support zone (941) of the guide support (940). The abutment is necessary to prevent the release of mechanical energy stored in the spring (350) compressed in the initial state. At the distal end thereof, the spring (350) rests against a laminar protrusion (950) of the lower casing of the housing, while the forces can also be taken up by the upper casing of the housing.

The cylindrical body (370) has the shape of a dovetail (378) diametrically opposite the leg (372) and of the same axial extent, the dovetail (378) also being able to be brought into axial abutment against a second support (942) of the guide support (940) to achieve a second retention of the spring (350) in the compressed state, radially symmetrical to the first abutment achieved by the leg (372). Of course, it is not strictly necessary for both stops to be located in the same axial plane, or even for two stops to be present, but this symmetrization promotes the creep resilience of the movable connection part (308) and also prevents the cylindrical body (370) from bowing in the guide support (940).

Thus, in the initial position, only rotational movement of the cylindrical body (370) is permitted in the guide support (940). This rotational movement can be achieved by engagement of the leg (372) with an extractable part (360) inserted in an opening in the lower casing (107) of the housing, the extractable part (360) being able to be extracted from the medical device in a direction orthogonal to the base of the lower casing (107) of the housing. The extractable part (360) has a cylindrical base (361) extended axially by a protuberance provided with a slot (362) wherein a pin (373) located at the radial end of the leg (372) and extending axially in the distal direction is inserted. The slot (362) has a cam profile (363) to guide a tangential movement of the radial end of the leg (372), when the extractable part (360) is extracted, causing the cylindrical body (370) to rotate. Note that the angular spread of the dovetail (378) and the leg (372) are selected so that both leave their support zone (941, 942) simultaneously, during the rotational movement imparted by the extractable part (360). The angular position in which the dovetail (378) and the leg (372) simultaneously leave their support zone (941, 942) is called the intermediate position and is shown in FIG. 25.

The lower casing (107) of the housing is lined with an adhesive membrane (365) covered by a cover (364) featuring a removal tab (367), this assembly protecting the area of the device affixed to the patient from any contamination. The cover must be removed by the patient before use so that the device can be applied to the skin and held in place by the adhesive membrane (365). The cylindrical base (361) of the extractable part (360) has a portion emerging from the lower casing (107) of the housing, which is attached to the cover (364) of the adhesive membrane (365), so that when the patient removes the cover (364) from the adhesive membrane (365), the latter also causes the extractable part (360) to be removed, thus setting the movable connection part (308) in rotational motion. The mechanical connection between the cover (364) of the adhesive membrane (365) and the extractable part (360) can be achieved in multiple ways and, in particular, by wedging the cover (364) by riveting into the extractable part (360) using a cap (366). To ensure a perfect seal, an O-ring (368) may be fitted between the cylindrical base (361) and the opening in the lower casing (107).

The guide support (940) also has a longitudinal groove (945) extending from the proximal end thereof to the distal end thereof. The longitudinal groove (945) is wider than the leg (372) and the dovetail (378), so that when both have left their support zones (941, 942) as a result of the rotational movement imparted by the extractable part (360), the cylindrical body (370) acquires an additional translational degree of freedom in its axial direction. A translational movement of the cylindrical body (370) towards the reservoir (203) is then impelled and constrained by the compressed spring (350), releasing its stored energy.

The cylindrical body (370) also has a radial protrusion (375) extending from the end of the dovetail (378) up to the proximal end of the cylindrical body (370), holding the hollow needle (315) to allow it to open out in the proximal direction. The hollow needle (315) may, for example, be held in the movable connection part (308) by an overmolding process, and the adapter part may be made from injection-molded plastic, but this is not however a limiting factor of the disclosure. Of course, this radial protrusion (375) also protrudes from the longitudinal groove (945) of the guide support (940) and has a sufficiently limited angular spread so as not to impede the rotational movement of the cylindrical body (370) imparted by the extractable part (360).

The translational movement of the cylindrical body (370) generated by the relaxation of the spring (350) forces the hollow needle (315) to penetrate the reservoir (203) through the septum thereof and thus fluidically connect the reservoir (203) with the pump system (200). Note that in the initial position, the hollow needle (315) is not collinear with the axis of symmetry of the reservoir (203) and it is only following completion of the first rotational movement to allow spring relaxation, that this alignment is obtained. The spring is carefully selected to ensure that the septum can be pierced with sufficient momentum and optionally another sterilization membrane (not shown).

Finally, the radial protrusion (375) of the cylindrical body (370) has an angled tab (376) at the distal end thereof, the end of the tab activating the switch (390) for powering the electronic system (310), activation of the switch taking place at the end of the axial movement of the cylindrical body (370) performed by relaxation of the spring (350).

In this way, the movable connection part (308) provides fluid connection between the reservoir (203) and the pump system (200) and the power supply to the transdermal drug injection device, in a single operation, this operation being induced by the intentional removal of the cover by the patient for the purpose of using the medical device. This combination judiciously limits any risk of mishandling and eliminates any risk of contamination right up to the point of use.

As can be seen in FIG. 21, the adhesive membrane (365) has a multiplicity of tabs (364) to facilitate the gripping and removal thereof by the patient.

1. A transdermal drug administration device comprising:
a. a housing;
b. a battery;
c. an injection system;
d. a movable connection part for providing fluid connection between the injection system and a reservoir for containing an active ingredient intended to be administered;
e. a pump system connected to the movable connection part and to the injection system by way of flexible pipes so as to form a fluid network, the pump system being motorized so as to enable a fluid supply to circulate within this network; and
f. an electronic system for controlling driving of the pump system, the electronic system including a mechanical switch for powering the pump system with the battery;
wherein the movable connection part has at least one degree of freedom relative to the housing on one side and the reservoir on the other to allow a movement, the movable connection part jointly controlling the fluid supply of the pump system and a power supply of the electronic system during the movement,
at least part of the movement of the movable connection part configured to be performed by a human mechanical action,
the movable connection part comprising a means for fluid connection between the reservoir and the pump system and providing disconnection at rest and fluid communication during the movement, and
the movable connection part comprising a means for activating an electrical power supply of electrical and electronic components of the transdermal drug administration device, providing disconnection at rest and supplying power during the movement.

2. The device of claim 1, wherein the reservoir comprises a vial, and the movable connection part is configured to receive the vial, the vial being removable from the transdermal drug administration device, the movable connection part configured to be moved according to the at least one degree of freedom when receiving the vial.

3. The device of claim 2, wherein the reservoir is connected to the movable connection part via an adapter part, previously connected to the reservoir, the movable connection part and the adapter part having complementary central pipes that fit together to form a sealed connection.

4. The device of claim 3, wherein the complementary central pipes form the sealed connection by a tapered fit.

5. The device of claim 4, wherein the tapered fit formed by the complementary central pipes is a luer fit.

6. The device of claim 5, wherein the movable connection part has a cylindrical body provided with a tapping, the adapter part having a hollow cylindrical protuberance into which the central pipe opens out, the hollow cylindrical protuberance having on an inner surface thereof a thread engaging with the tapping to provide a tight fitting of the complementary central pipes by screwing an adapter part with the movable connection part, the sealed connection being formed beyond a nominal tightening torque.

7. The device of claim 6. wherein the at least one degree of freedom of the movable connection part for activating the mechanical switch is a rotational degree of freedom achieved by guiding the movable connection part in the lower casing of the housing, a movement path of the movable connection part being angular, and wherein the movable connection part has a partial annular protuberance axially extending the cylindrical body over a limited angular portion, the partial annular protuberance being capable, at an end of travel, of activating the mechanical switch located in radial proximity to the movable connection part.

8. The device of claim 7, wherein the movement path of the movable connection part is traversed by a rotational movement leading to screwing of the adapter part with the movable connection part.

9. The device of claim 8, wherein the movement path of the movable connection part, according to the at least one degree of freedom, has a notch at the end of travel to lock the movable connection part in a final position, the notch requiring excess torque to be applied and reach the final position, the mechanical switch being activated in the final position.

10. The device of claim 9, wherein the excess torque required to pass the, notch and reach the final movement position of the movable connection part is greater than a nominal tightening torque for screwing an adapter part with the movable connection part, thereby forming a sealed fit of the complementary central pipes.

11. The device of claim 10, wherein the movable connection part has two cylindrical axial protuberances extending from the cylindrical body, the axial protuberances extending into the lower casing of the housing and simultaneously coming into abutment against beads during a course of the movement path of the movable connection part, the beads being protrusions of flexible beams integral with the lower casing, the abutment position of the axial protuberances against the beads being able to be exceeded by elastic deformation of the flexible beams, being elastically deformable, to reach the final movement position of the movable connection part, the notch thus being achieved by the elastic deformation of the flexible beams.

12. The device of claim 11, wherein the adapter part is screwed to the movable connection part by a user.

13. The device of claim 12. wherein the elastic deformation and release of the flexible beams, when the user applies excess torque to pass the notch, provides haptic information signaling activation of the mechanical switch and, therefore, of the device.

14. The device of claim 9, wherein the movable connection part has a radial protuberance and a partial annular protuberance, the final position of the movable connection part being achieved by bringing the radial protuberance and partial annular protuberance respectively into contact with complementary stops of the lower casing of the housing.

15. The device of claim 1 wherein the movable connection part includes a hollow needle connected to a flexible pipe, the hollow needle being configured to be inserted into the reservoir upon a translational movement of the movable connection part to reach a final state.

16. The device of claim 15, wherein the translational movement is induced by a spring.

17. The device of claim 16, wherein, in an initial state, the movable connection part has no translational degree of freedom, but only one translational degree of freedom, and wherein the spring is in a compressed state.

18. The device of claim 17, wherein a first rotational movement of the movable connection part is performed by a mechanical action of a patient to bring the movable connection part into an intermediate state in which the movable connection part acquires the only one translational degree of freedom allowing the hollow needle to be inserted into the reservoir.

19. The device of claim 2, wherein the movement path of the movable connection part, according to the at least one degree of freedom, has a notch at an end of travel of the movable connection part to lock the movable connection part in a final position, the notch requiring excess torque to be applied and reach the final position, the mechanical switch being activated in the final position.

20. The device of claim 6, wherein the movable connection part has a radial protuberance and a partial annular protuberance, a final position of the movable connection part being achieved by bringing the radial protuberance and partial annular protuberance respectively into contact with complementary stops of the lower casing of the housing.
